(19) United States
(12) Patent Application Publication
(10) Pub. No.: US 2005/0277876 A1
(43) Pub. Date: Dec. 15, 2005

(54) Medical device guiding system

(75) Inventor: Scott William Hayden, Menomonie, WI (US)

Correspondence Address: Scott Hayden, 6 Center Street, Sea Bright, NJ, 07760, US

(73) Assignee: Scott Hayden

(21) Appl. No.: 11/089,175

(22) Filed: Mar. 25, 2005

Related U.S. Application Data
(60) Provisional application No. 60/578,391, filed on Jun. 10, 2004.

(51) Int. Cl. A61M 31/00

(52) U.S. Cl. 604/95.04

(57) ABSTRACT

The invention disclosed herein is a steering system that can be attached to a catheter, guidewire or obturator that consists of polymeric tubing with a center lumen and at least three off-axis lumens evenly spaced around the circumference of the device shaft. The distal sections of the off-axis lumens are formed to induce curvature of the tip of the device by a physician controlled pressure source, which may be foot activated. Forming the individual lumens to induce curvature of the shaft of the device in a certain direction is done by making one side of each lumen significantly longer, such as with a one sided corrugated configuration. These embodiments induce curvature in predetermined directions when pressurized.

### STATEMENT REGARDING FEDERALLY SPONSORED RESEARCH AND DEVELOPMENT

Not applicable

### CROSS-REFERENCE TO RELATED APPLICATIONS

This application is a continuation of U.S. provisional patent application No. 60/578,391, filed on Jun. 10, 2004.

### REFERENCES CITED

| U.S. Pat. No. | Classes | Inventor |
|---|---|---|
| 3,773,034 | 604/95.01; 600/434 | Burns et al. |
| 4,685,473 | 600/585; 600/587; 604/95.03 | Karcher, et al. |
| 4,838,859 | 604/95.03 | Strassmann; Steve |
| 4,906,230 | 604/95.03; 600/152 | Maloney, et al. |
| 4,909,787 | 604/95.03; 604/913; 606/194 | Danforth; John W. |
| 4,983,165 | 604/95.03 | Loiterman; David A. |
| 5,123,421 | 600/585; 600/434; 604/95.01 | Sinofsky; Edward L |
| 5,308,323 | 604/95.03; 606/192; 606/194 | Sogawa; Ichiro |
| 5,314,428 | 604/95.03; 600/434 | Marotta; Louis C. |
| 5,364,353 | 604/95.03; 600/116; 600/140 | Corfitsen; Mogens T |
| 6,165,123 | 600/152; 600/114; 600/143; | Thompson; Robert Lee |
| | 600/159 | |
| 6,338,725 | 604/95.04; 604/96.01; | Hermann; George D. |
| | 604/164.01; 604/167.01; | |
| | 604/523; 604/537; 604/912 | |
| 6,612,999 | 600/585 | Brennan; Lawrence |

### BACKGROUND OF THE INVENTION

The present invention relates generally to a medical device guiding system, and more particularly to a multi-directional steering system for intravascular catheters, guidewires and obturators that can be controlled with a fluid pressurization system.

In general, the term catheter as used in this application includes a wide variety of devices in the fields of cardiology, radiology and neuroradiology. For example, guide catheters provide a conduit that may be used to deliver a device, such as an angioplasty balloon, stent, lead or coil, to areas in the heart, brain or peripheral vasculature. Other catheters may be used to deliver fluid, administer drugs, radiation, thermal therapy, RF ablation, cryotherapy, record electrical impulses or produce an electric stimulus.

A physician must navigate catheters, guidewires and obturators through highly curved paths to reach a target site. In many cases, a physician must also orient the tip of the catheter in a certain direction after reaching the target location in order to complete the procedure. Devices often fail to reach a target location due to combined stresses from bending, axial, and torsional loads. A method of orienting the tip of these devices, without twisting the shaft, would reduce the combined stresses in the shaft and lead to better performance. Also, a method of changing the stiffness of the shaft during a procedure may prevent shaft prolapse and/or increase back-up support, further increasing device performance.

Catheters having pull wires to deflect the tip are known. However, these devices often don't produce sufficient turning radius and are too large and rigid for many procedures. Catheters having inflatable sections located on the distal section of the catheters are also known. However, these embodiments have significant limitations. These limitations include being too complicated to use, too big in diameter, too expensive to manufacture, or provide insufficient turning radius. A system that could overcome these limitations would be desirable. In certain medical procedures, a system with a combined means of deflecting the device tip and providing an axial forward force near the tip, would be highly desirable. Giving the physician the option of controlling the tip orientation by foot would also be desirable.

### BRIEF SUMMARY OF INVENTION

The invention disclosed herein is a steering system that can be attached to a catheter, guidewire or obturator that overcomes limitations of prior art. The system consists of polymeric tubing with a center lumen to accommodate the catheter, guidewire or obturator and at least three off-axis lumens evenly spaced around the circumference of the device shaft Each off-axis lumen is open on the proximal end to allow selective fluid pressurization in each lumen and closed on the distal end to prevent fluid from entering the vessel. The distal sections of the lumens are formed to induce curvature of the tip of the device by a physician controlled pressure source, which may be foot activated. Each lumen has a separate control means for selective pressurization of each lumen. The system provides at least 45 degrees of bending. Pressurization of more than one lumen simultaneously allows bending at the bisectrices of individual lumen bending directions. Pressurization of all lumens equally increases the stiffness of the shaft, which may be done without deflecting to tip in the preferred embodiment.

Forming the individual lumens to induce curvature of the shaft of the device in a certain direction can be done by making one side of each lumen significantly longer, such as with a one sided corrugated configuration. Either the inside or outside of the lumens may be made longer. In another embodiment, lumens may consist of preformed configuration, such as a shaped balloon. These embodiments induce curvature in predetermined directions when pressurized.

In another embodiment, the distal end of the lumens may include at least one section that expands radially outward significantly more than the other sections of the lumen. This may allow the physiologic blood flow to produce an axially forward force on the device shaft and additional bending force.

### BRIEF DESCRIPTION OF THE SEVERAL VIEWS OF THE DRAWING

FIG. 1: Side view of the distal end of the system bending catheter in multiple directions

FIG. 2: Side view of system navigating a medical device through vascular bifurcation

FIG. 3: Side view of system steering tip of catheter into aneurysm

FIG. 4: Side view of system deflecting tip of catheter in heart chamber

FIG. 5: Cross-sectional view of multi-lumen tubing for system

FIG. 6: Cross-sectional side view of system without pressurization mounted on catheter with external wall corrugated shaped

FIG. 7: Cross-sectional side view of system mounted on catheter with internal wall corrugated shaped

FIG. 8: Cross-sectional side view of system mounted on catheter with distal wall consisting of shaped balloon

FIG. 9A: Side view of system mounted on catheter with one part of distal section expanded significantly more than other sections of inflation lumen showing axial forward force from blood flow.

FIG. 9B: Side view of system mounted on catheter with multiple parts of distal section expanded significantly more than other sections of inflation lumen showing axial forward force from blood flow.

### DETAILED DESCRIPTION OF THE INVENTION

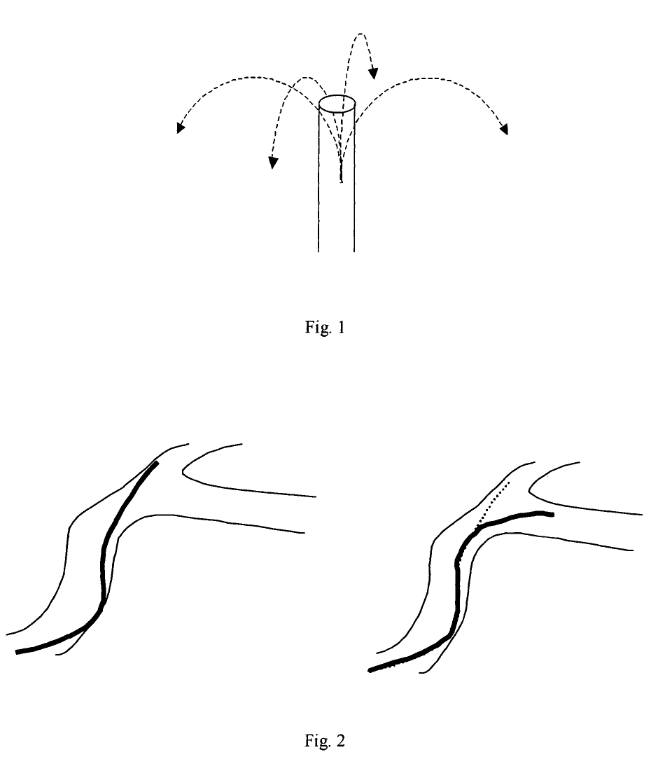

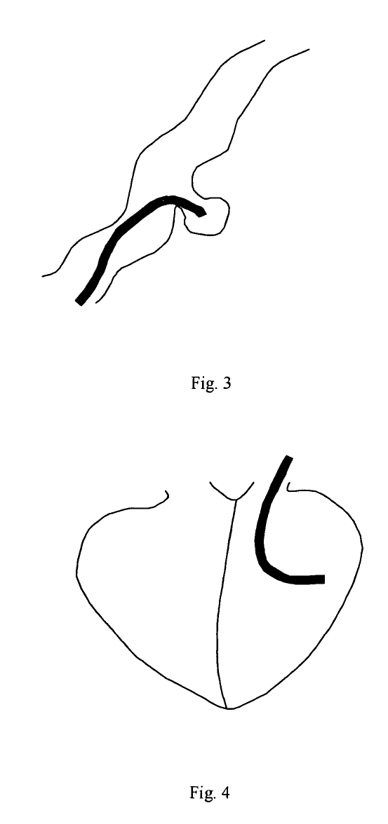

The invention disclosed herein is intended to provide physicians with a tool to allow better treatment options for patients with disease to the heart, brain and peripheral vascular system. It may be added to the external surface of a catheter, guidewire or obturator. The system can be manufactured as part of the catheter, guidewire or obturator device or attached to the device in the catheterization lab. Pressurizing the inflation lumens causes the distal end of the device to curve in different directions as shown in FIG. 1. The amount of curvature increases with increased pressure until maximum curvature is reached. Maximum deflection will be at least 45°. This will help a physician perform the procedure in several ways, including navigate through highly curved vessels as shown in FIG. 2, or point the tip towards the intended target such as in a aneurysm as shown in FIG. 3 or heart chamber wall as shown in FIG. 4. Below is described the preferred embodiments of this guiding system.

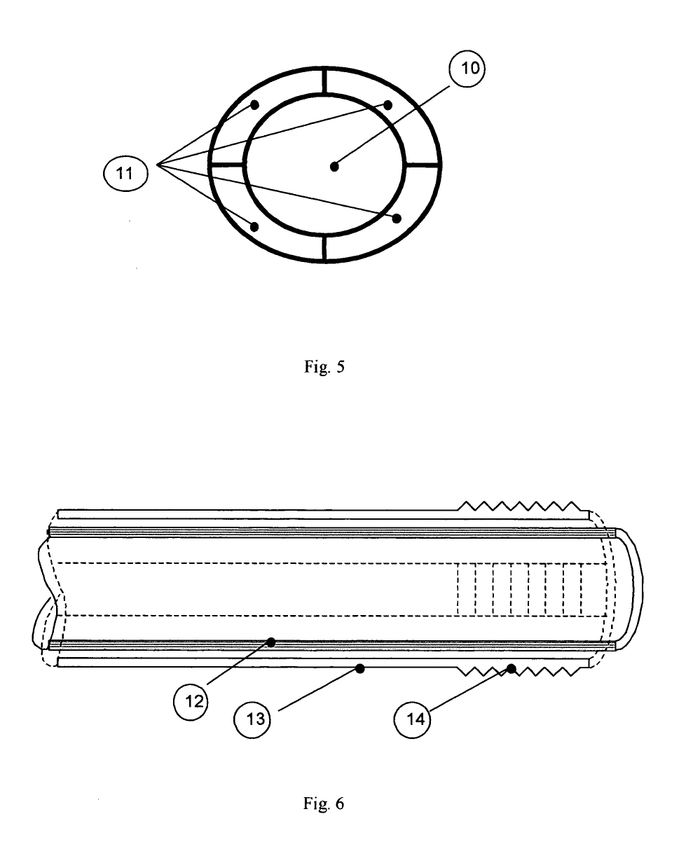

The main body of the preferred embodiment consists of a multi-lumen extruded polymer with a shore durameter between 80A and 75D. Preferred polymers include polyurethane, PEBA, or amide; Elastic polymers with similar properties or combined polymer shafts may also be used. The multi-lumen tubing would consist of a center lumen surrounded circumferentially by at least three inflation lumens. FIG. 5 shows the center lumen 10, which accommodates the catheter, guidewire or obturator, surrounded by four off-axis inflation lumens 11. The number of inflation lumens would be at least three to provide 360° turning range but fewer than 25. For most applications, the preferable number of lumens would be in the range of three to eight. The tubing may also consist of two or more multi-lumen tubes bonded together. For example, a stiffer multi-lumen material may be used on the proximal portion of the shaft and then bonded by heat or adhesive to a more flexible multi-lumen tubing located on the distal end of the system. The center lumen inner diameter at the distal section would have a range of 0.25 to 3.5 millimeters. The outer diameter of the distal section of the system with internal pressure in the inflation lumens would have a range of 0.3 to 4.0 millimeters.

Selective pressurization of at least one inflation lumen produces a bending force on the device located within the center lumen. The bending force is produced by differential expansion within the inflation lumen walls. The bending section is near the distal tip, starting within five centimeters of the distal end. The length of the maximally inflated deflection section will vary between 0.25 and 25 centimeters. For most applications, the preferred length of this section will be between 1 and 15 centimeters when maximally pressurized. Maximum inflation pressure is between 2 and 50 atmospheres. Inflating adjacent lumens causes the system to curve at bisectrices. Three preferred embodiments of this concept are described herein, with each having slightly different advantages.

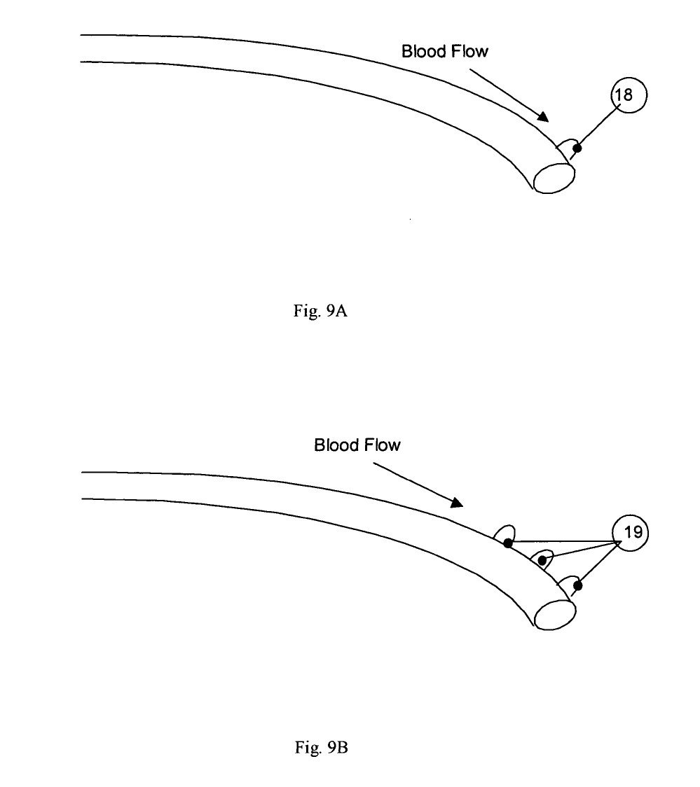

In the first preferred embodiment, shown in FIG. 6 attached to a catheter shaft 12, the outside surface of the inflation lumens 13 have a corrugated type shape 14. Pressurization of one lumen produces the system to curve on the opposite side of the corrugated lumen. The corrugation type shape of one surface of tubing can be made using multiple techniques. One technique involves placing mandrels in the center and inflation lumens, pressing together axially the section of the tubing to be corrugated, and then heat to set in shape. A secondary process involves placing a heated mandrel in the center lumen to re-flow the inner surface, thereby reducing or removing the corrugated type shape. Alternatively, the outer surface can be formed in to a corrugated shape by placing a mandrel in the center lumen and a female die around the exterior surface, then pressurizing the inflation lumens to conform the polymeric outer surface to the metallic female die surface. This embodiment allows the incorporation of a radially extended section 18 as shown in FIG. 9A. Multiple radially extended sections 19 may also be incorporated as shown in FIG. 9B. The size and shape of the radially extended section can be formed using the female die technique similar to that described above.

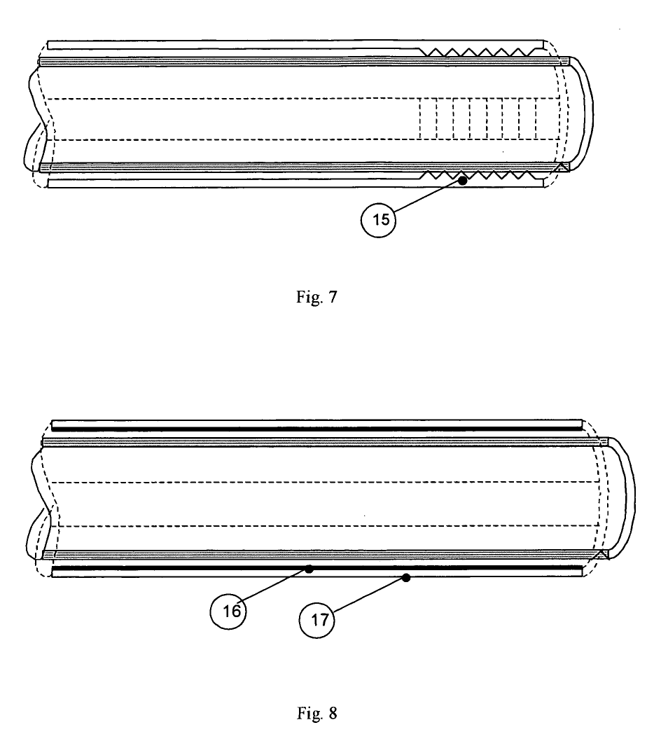

In the second preferred embodiment, shown in FIG. 7, the inside surface 15 of the inflation lumen has a corrugated type shape. Pressurizing one lumen produces curvature on the same side as the inflation lumen. The corrugation type shape of one surface of tubing can be made using multiple techniques. One technique involves placing mandrels in the center and inflation lumens, pressing together axially the section of the tubing to be corrugated, and then heat to set in shape. A secondary process involves placing a heated smooth surfaced die over the tubing to reduce or remove the corrugated type shape from the outer surface of the inflation lumens. Alternatively, the inside surface can be formed in to a corrugated type shape by placing a corrugated shaped mandrel in the center lumen and a smooth die around the exterior surface, then pressurizing the inflation lumens to conform the inner surface of the lumens to the outer surface of the mandrel and set shape with heat. This embodiment doesn't allow the incorporation of a radially extended section as shown in FIGS. 9A and 9B.

The third preferred embodiment, shown in FIG. 8, discloses a system whereby the distal end of the inflation lumens are formed into shaped tubes. Pressurizing one lumen produces axial curvature in a predetermined direction, depending on how the inflation lumens are shaped. In one design, the lumens may have a more compliant inner surface 16 which produces deflection on the same side as the lumen. Alternatively, the lumens may have a more compliant outer surface 17 which produces deflection on the opposite side as the lumen. Methods to produce a lumens with differential compliance of the inside surface relative to the outside surfaces, includes annealing only one surface, stretching one surface more than the other, re-lowing one surface, using thicker material on one side of each lumen, or combining two or more of the methods. Annealing and reflowing one surface may be done with heated dies or mandrel and pressurizing the lumen. The dies and mandrels may be straight or curved, depending on the type of shape and amount of curvature desired. One preferred method for differential stretching is to secure one side of the lumen and then stretching the other by pressurizing the lumen. This embodiment can also be used to create compound curves, such as loop that creates contact around the internal surface of a blood vessel wall. The embodiment of this system that curves opposite the side of the lumen may incorporate a radially extended section as shown in FIGS. 9A and 9B.

These three embodiments disclosed herein may also incorporate radial restraint for the outer surface. For example, the outside surface may have restrains that increase its burst strength and provide a means to increase the system deflection. These restraints may be in the form of a single lumen tube, spiral wrap, webbed tubing, a series of spaced circular sections attached to the outer surface of the system. The outer surface of distal section of the multi-lumen tubing may be covered with a lubricious outer layer material, such as ePTFE, or coating, either hydrophilic or hydrophobic.

Hubs with luer fittings are connected to the proximal end of the inflation lumens. This permits quick connection of the pressure source. The pressure source may consist of a series of foot activated piston-type pumps, such as a syringe. A doctor could then step on one or more piston to create pressure in the lumen or lumens. A relief valve may be placed inline between the pump and hub of the inflation lumen to prevent rupture caused from too high input pressure. A foot-activated, instead of hand activated, system may free the physician's hands for other tasks commonly performed when diagnosing or treating a patient. An automated pump system could also be used but would be more expensive.

The invention disclosed herein differs from prior art in several ways. It may be added to a catheter, guidewire or obturator in the catheterization lab and doesn't need to be manufactured as part the device. Therefore, physician may attempt to complete the procedure without the guiding system initially. If unsuccessful, the system may be added to the device in the catheterization lab. Alternatively, this system can also be permanently attached to the catheter, guidewire or obturator during manufacturing.

1. A medical device steering system comprising:
an elongate multi-lumen tube with an open center lumen extending from a proximal end to a distal end of its length;
at least three off-axis lumens around circumference of said center lumen with differential expansion section and proximal end open to allow fluid communication and distal end closed to prevent fluid from entering vessel;
at least three pressurization sources in fluid communication with said off-axis lumens;

2. A device as in claim 1 wherein said off-axis lumens are comprised of amide, PEBA, or urethane.

3. A device as in claim 1 wherein said differential expansion section is located within 25 centimeters of distal tip.

4. A device as in claim 1 wherein said differential expansion section is capable of producing deflection of at least 45 degrees.

5. A device as in claim 1 wherein said multi-lumen tube is attached to a catheter shaft or guidewire or obturator.

6. A device as in claim 1 wherein said pressurization sources are piston-type pumps.

7. A device as in claim 1 wherein said differential expansion section is covered with a radial restraint.

8. A device as in claim 1 wherein distal end of said multi-lumen tubing is covered with lubricious material such as ePTFE tubing.

9. A device as in claim 1 wherein distal end of said multi-lumen tubing is coated with hydrophobic or hydrophilic material.

10. A device as in claim 1 wherein distal end of said differential expansion section incorporates at least one radially extended section.
